(19) United States
(12) Patent Application Publication
(10) Pub. No.: US 2024/0299767 A1
(43) Pub. Date: Sep. 12, 2024

(54) DISTRIBUTED DOSING IN FREE-SPACE DELIVERY OF PHOTO-BIO MODULATION IRRADIATION

(71) Applicant: Seaborough Life Science B.V., Amsterdam (NL)

(72) Inventors: Charlie MINTER, Amsterdam (NL); Michael KRAMES, Kentfield, CA (US)

(21) Appl. No.: 18/281,989

(22) PCT Filed: Mar. 15, 2022
(86) PCT No.: PCT/EP2022/056745

§ 371 Date: Sep. 14, 2023

(30) Foreign Application Priority Data
Mar. 26, 2021 (US) 63166331

(51) Int. Cl. A61N 5/06

(52) U.S. Cl. CPC A61N 5/0613; A61N 2005/0626; A61N 2005/0643; A61N 2005/0651; A61N 2005/0663; A61N 2005/0665

(57) ABSTRACT

A lighting system comprising a first light source adapted to emit light substantially only in a first predetermined spectrum in a range from 600 nm to 1400 nm, and a driver circuit arranged to provide a first pulsed current to the first light source for producing the light in the first predetermined spectrum. The driver circuit is adapted to generate multiple pulses of the first pulsed current during a first period and no pulses of current during a second period, the first period and the second period alternating with each other, and the first pulsed current has a first pulse frequency and a first duty cycle during the first period, the first pulse frequency being 100 Hz or higher, and the first duty cycle being 0.5 percent or above.

### TECHNICAL FIELD

The invention relates generally to lighting, and more particularly to a lighting apparatus, a lighting system, and a method for providing red or near-infrared radiation to induce a photobiomodulation (PBM) response.

### BACKGROUND

Exposure of certain amounts of red (R) to near-infrared (NIR) radiation to living organisms has been shown to induce biological and/or biochemical responses which lead to beneficial effects such as stimulating healing, relieving pain, and reducing inflammation. To employ this technique, called photobiomodulation (PBM), the conventional approach has been to deliver the required radiation through a dedicated device applied directly to or very near the surface of the skin of the person or animal being treated.

The concept of delivering PBM effects via free-space radiation within the context of a general purpose illumination system has been proposed by the Applicant of the present application. To deliver the required irradiated intensity levels, high peak pulsing of the R/NIR radiation sources was employed, as described for example in PCT publication WO 2020/119965 A1. Various ranges of pulse-widths and frequencies are described to deliver a targeted PBM dose expected to provide beneficial effects. Within these ranges, embodiments are described such that visible light from the illumination system would not produce perceptible flicker to the human eye. This goal was achieved in part by exploiting the fact that the human eye is not very sensitive to deep-red or NIR light.

Electronic imaging systems such as digital cameras have sensors (e.g., CMOS or CCD sensors) that are more sensitive to deep-red and NIR radiation than the human eye. This can lead to situations in which a PBM illumination system provides pulsed radiation that, while not directly visible as flickering to the human eye, will nevertheless appear to flicker when perceived through an electronic imaging system. This “image flicker” can be annoying and disruptive to important tasks, such as patient health monitoring in a hospital setting, for example.

Thus, it is desired to provide a method for free-space delivery of effective PBM radiation, which does not suffer from significant levels of human-visible flicker or image flicker. The present invention aims to provide a solution.

### SUMMARY OF THE INVENTION

It would be desirable to provide an apparatus that is easy to use, energy efficient, cost effective and yet emits an amount of radiation sufficient to induce PBM response.

According to a first aspect of the present disclosure, a lighting system is provided comprising a first light source adapted to emit light substantially only in a first predetermined spectrum in a range from 600 nm to 1400 nm, and a driver circuit arranged to provide a first pulsed current to the first light source for producing the light in the first predetermined spectrum. The driver circuit is adapted to generate multiple pulses of the first pulsed current during a first period and no pulses of current during a second period, the first period and the second period alternating with each other, and the first pulsed current has a first pulse frequency and a first duty cycle during the first period, the first pulse frequency being 100 Hz or higher, and the first duty cycle being 0.5 percent or above.

The driver circuit may be adapted to generate the first pulsed current having an amplitude of the pulses which increases for successive ones of the pulses during a first portion of the first period, and decreases for successive ones of the pulses during a last portion of the first period. The driver circuit may be adapted to generate the first pulsed current having an amplitude of the pulses which is substantially constant during a second portion of the first period. The driver circuit may be adapted to generate the first pulsed current having a pulse-width of the pulses which increases for successive ones of the pulses during a first portion of the first period, and decreases for successive ones of the pulses during a subsequent portion of the first period.

The driver circuit may be adapted to generate the first pulsed current having a pulse frequency which is a multiple of 24 pulses per second and/or 30 pulses per second, and/or having a pulse frequency which is a multiple of a mains power supply frequency, and/or having a pulse frequency being a multiple of a frame rate of an imaging device capable of recording images and/or video. The driver circuit may be adapted to generate the first pulsed current having a width of the pulses of 0.05 ms or more, and/or having a period between pulses of 0.05 ms or more.

In another aspect of the disclosure, a lighting system is provided comprising a first light source adapted to emit light substantially only in a first predetermined spectrum in a range from 600 nm to 1400 nm, and a driver circuit adapted to provide a first current to the first light source for producing the light in the first predetermined spectrum. The driver circuit is configured to provide the first current during a first period and not during a second period, the first period and the second period alternating with each other, and the driver circuit is configured to gradually increase the amplitude of the first current during a first portion of each first period, and gradually decrease the amplitude of the first current during a last portion of each first period. The driver circuit may be configured to maintain the first current at a substantially constant amplitude during a second portion of each first period, the second portion occurring between the first portion and the last portion of each first period.

The lighting systems according to the first or second aspect may be configured so that the ratio between the first period to the second period may be 1:10 or less, and may be configured to generate a irradiation intensity at an average distance of between 0.2 and 5 m from the first light source of 1 mW/cm² or more, preferably between 0.4 and 50 mW/cm², and more preferably between 1 and 15 mW/cm². The lighting systems may be configured so that the irradiation intensity at an average distance of between 0.2 and 5 m from the first light source is sufficient to induce a photobiomodulation effect in a human. The delivered dose over 8 hours at an average distance of between 0.2 and 5 m from the first light source may be between 0.01 and 50 J/cm², and preferably between 0.1 and 10 J/cm².

The lighting systems according to the first or second aspect may further comprise a second light source adapted to emit white light suitable for general illumination, wherein the second light source is adapted to emit at least 250 lumens, preferably at least 1000 lumens, more preferably at least 2000 lumens when operating. The white light emitted by the second light source may be directed onto one or more reflectors so that the white light is emitted from the lighting system having a radiation pattern with a full-width-at-half-power angle of 2×23 degrees or more. The light emitted by the first light source may be emitted from the lighting systems having a radiation pattern with a full-width-at-half-power angle of 2×45 degrees or less.

The lighting systems may comprise a luminaire, wherein the first and second light sources and the one or more reflectors are installed in the luminaire. The lighting systems may comprise a lamp for lighting a work space, wherein the first and second light sources and the one or more reflectors are mounted in the lamp, the lamp being adapted to direct the white light from the second light source onto a work space and to direct the light from the first light source onto a user.

### BRIEF DESCRIPTION OF THE DRAWINGS

Embodiments will now be described, by way of example only, with reference to the accompanying schematic drawings in which corresponding reference symbols indicate corresponding parts, and in which:

FIG. 1 is a schematic diagram of a lighting system in accordance with an embodiment of the present disclosure;

FIG. 2 is a graph indicating sensitivity to various wavelengths of light;

FIG. 3 shows images of an NIR LED array pulsed at various frequencies;

FIG. 4 is a diagram showing distribution of illumination light and PBM radiation from an exemplary luminaire;

FIG. 5 is a diagram showing distribution of illumination light and PBM radiation from another exemplary luminaire;

FIG. 6 is a schematic diagram of an embodiment of a lighting system in a desktop lamp;

FIGS. 7A and 7B are diagrams of a pulsing current for driving a PBM light source;

FIG. 8 is diagram of a pulsing current for driving a PBM light source in accordance with an embodiment of the present disclosure;

FIG. 9A is diagram of a pulsing current for driving a PBM light source in accordance with another embodiment of the present disclosure;

FIG. 9B is diagram of a pulsing current for driving a PBM light source in accordance with another embodiment of the present disclosure;

FIG. 10A is diagram of a non-pulsing current for driving a PBM light source; and

FIG. 10B is diagram of a non-pulsing current for driving a PBM light source in accordance with an embodiment of the present disclosure.

The figures are intended for illustrative purposes only, and do not serve as restriction of the scope of protection as laid down by the claims.

### DESCRIPTION OF ILLUSTRATIVE EMBODIMENTS

The following is a description of certain embodiments of the invention, given by way of example only and with reference to the drawings.

A free-space radiation system is provided to deliver radiation in the red (R) (600-700 nm) and/or near-infrared (NIR) (700-1400 nm) spectrum, and capable of achieving the irradiated intensity levels on target subjects to induce a meaningful PBM response in the target subject, for example as described in International Application No. PCT/EP2020/083093 and International Publication No. WO 2020/119965, both of which are incorporated by reference herein in their entirety.

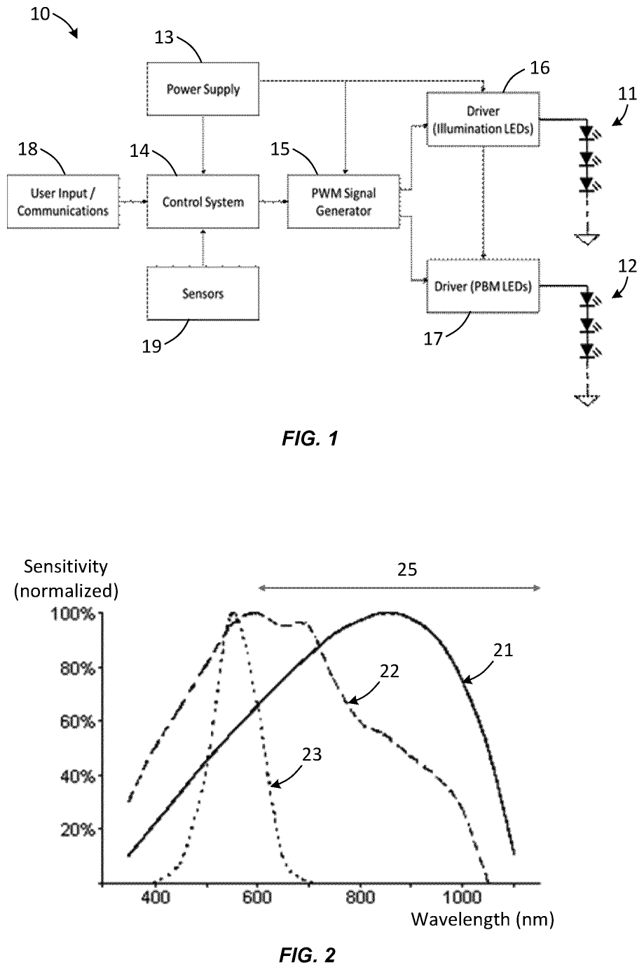

FIG. 1 is a schematic diagram of an embodiment of such a system. The system 10 includes a power supply 13 which delivers power to a control system 14, pulse-width modulation (PWM) signal generator 15, and to driver circuit 17 for driving one or more light-emitting diodes (LEDs) 12 which are the primary radiation source(s) for PBM. The peak emission wavelengths for the PBM LEDs 12 are within the R or NIR spectral ranges, or a combination thereof. The system may also include driver circuit 16 for driving light sources for illumination, such as visible-spectrum LEDs 11 for generating white light. The radiation output levels of the light sources 11 and LEDs 12 are determined by the control system in combination with the PWM signal generator 15 and driver circuits 16, 17. The control system 14 may take inputs 18 from users, through control devices (such as dimmers) or via communications through a network or app. Also, sensors 19, such as proximity sensors or ambient light sensors, may give inputs to control system 10.

The PBM LEDs 12 may be integrated amongst the illuminations light sources 11 (which may be LEDs, but may include conventional light sources such as incandescent, fluorescent, or electric-discharge lamps), or contained separately. Also, the illumination light sources 11 and PBM LEDs 12 may have separate driver electronics 16, 17 as shown in FIG. 1, so that they may act independently, or may share driver electronics.

Driving the PBM LEDs 12 with a pulsing current is effective because of the threshold effect involved in inducing a PBM effect in a person or other subject target. For example, it is generally accepted that an irradiation intensity of >1 mW/cm² at the skin's surface is required to induce a meaningful PBM effect. As a comparison, continuous illumination to achieve 500 lux (a typical target for office lighting) using white light results in less than 0.2 mW/cm² irradiation intensity. Due to the higher intensity requirement for inducing PBM, it can be prohibitive from a cost and energy use point of view to continually deliver radiation meaningful for a PBM effect over long periods. Instead, it can be advantageous to pulse a R and/or NIR emitter (such a PBM LEDs 12) to overcome the irradiation intensity threshold described above and deliver a meaningful PBM dose, while maintaining an average power dissipation that avoids overheating the emitter in the case that sustained high power density operation of the emitting device would cause thermal management challenges (such as overheating of the emitter) and possibly PBM radiation output degradation and/or reduced operating lifetime of the emitter.

Alternatively, the PBM irradiation intensity may be increased by focusing the emitted light into a radiation pattern narrower than that required for general illumination. That is, unlike for general illumination, in which large areas of a built environment require some level of irradiation in order to be seen, the PBM radiation need only be directed to the subject target, which may be located in a designated location, such as in a chair at a desk, or in a bed in a hospital room. This allows focusing or directing the PBM radiation, to create a higher irradiated intensity over a smaller area while using less light, according to the inverse-square law of electromagnetic radiation. For example, compared to a Lambertian emission (aka “cosine” distribution), which might be useful for general illumination, the possible increase in intensity as the radiation pattern full-width-at-half-power (FWHP) angle narrows is shown in Table 1 below:

| | | Relative |
|---|---|---|
| Radiation | | Irradiated |
| Pattern | FWHP | Intensity |
| Lambertian | 2 × 60° | ~1× |
| Flood | 2 × 38° | ~2× |
| Narrow Flood | 2 × 23° | ~5× |
| Spot | 2 × 10° | ~25× |
| Narrow Spot | 2 × 5° | ~100× |

The above effect can be used to reduce the total amount of power required to achieve the target irradiated intensity levels necessary for a PBM effect. Using this approach, it may be possible to exceed the irradiated intensity thresholds for PBM using LEDs in continuous-wave mode, while still managing the thermal situation properly. In this case, it may not be necessary to pulse the PBM LEDs 12.

Of course, increase in irradiated intensity levels may also be achieved by moving the radiation source closer to the target subject, or vice versa.

As a comparative example, a PBM radiation system may be selected to provide PBM dosing by continuously pulsing its PBM LEDs so that an ideal dose may be experienced for a person that experiences this radiation over a selected duration, such as an eight-hour work shift. For example, the PBM LEDs may be pulsed on for a period of 8 ms, and at a frequency of 10 Hz. Under the proper irradiation intensity levels, e.g., 1-10 mW/cm², a dose of 2.3-23 J/cm² may be achieved. Unfortunately, while this comparative example may not result in human-visible flicker of the PBM LEDs, it can produce image flicker.

Sensors used in electronic imaging equipment such as video recorders and cameras, including cameras typically built into smartphones, are more sensitive to infrared light than the human eye. FIG. 2 shows a normalized sensitivity of a typical CCD sensor 21, a typical CMOS sensor 22, and a typical human eye 23 plotted against wavelength of light from an emitter. As can be seen, CCD and CMOS sensors are more sensitive than the human eye to light in the wavelength region between 600 nm and out to beyond 1000 nm. Light emitted in this wavelength region from a PBM radiation system can interfere with the imaging function of these devices. In particular, under certain conditions, pulsing radiation in this wavelength regime can result in image flicker which may be recorded by such imaging devices producing undesired results.

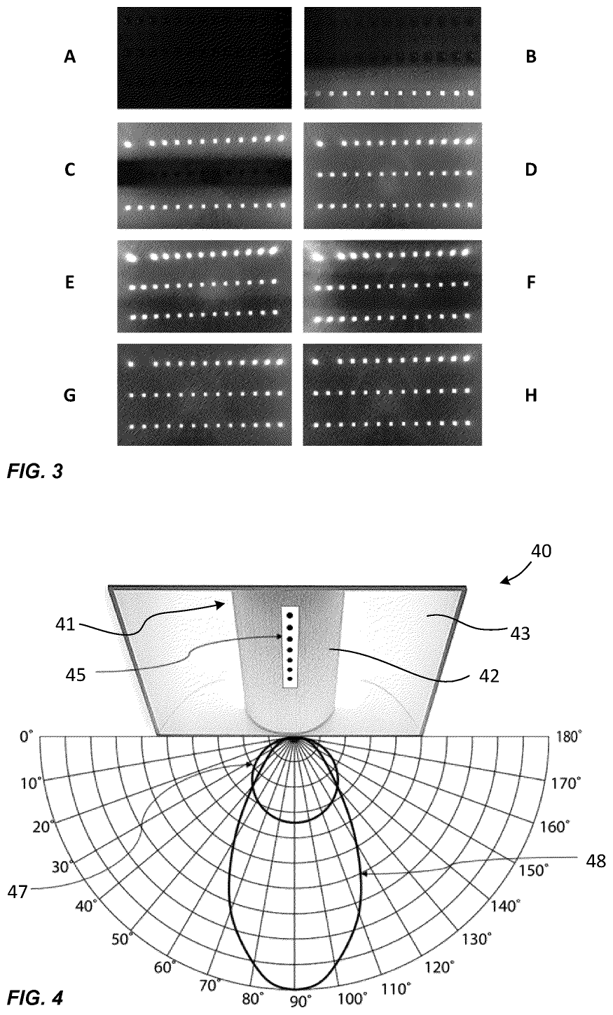

FIG. 3 shows some examples of image flicker from a pulsed NIR LED array, using screenshots from video taken with a typical smartphone camera pointed at the NIR LED array. In a first example, the NIR LED array was pulsed on for a period of 8 ms with a pulse frequency of 10 Hz. This resulted in image flicker being clearly observed. Image A of FIG. 3 is a resulting screen-shot, the image being dark because the pulsed LEDs are “off” during the entire frame cycle of the smartphone camera.

Frequency may be increased in an attempt to reduce the image flicker effect, while compensating the pulse duration appropriately to maintain the same dosing. The inventors explored this effect, as shown further in FIG. 3. The images shown are video screen-shots generated from a smartphone camera of an NIR LED array pulsed at variable frequencies from 10 to 320 Hz at ambient intensity levels high enough to be observed: image A at 10 Hz, B at 20 Hz, C at 25 Hz, D at 30 Hz, E at 50 Hz, F at 80 Hz, G at 100 Hz, and H at 320 Hz. Image flicker is observed at 10 Hz, 20 Hz, 25 Hz, 50 Hz, and 80 Hz. The image flicker appears as horizontal stripes in the screen shot images. It is notable that image flicker is not observable at 30 Hz, which corresponds to the frame rate of the smartphone camera used for the imaging in FIG. 3. It is also notable that no observable image flicker is observed at 100 Hz or higher frequencies.

According to one aspect of the present invention, it is desirable to choose a pulse frequency of 100 Hz or higher for pulsing the PBM LEDs 12 to avoid noticeable image flicker. According to another aspect of the present invention, it is desirable to choose a pulse frequency for the PBM LEDS 12 which is a multiple of the typical frame rates of commonly used imaging equipment, such as smartphone video cameras, and particularly a multiple of the frame rate of the imaging equipment to be used in the vicinity of the PBM luminaire or luminaires. For example, if the frame rate of the relevant imaging equipment is 30 frames per second (fps), suitable choices for the pulse frequency of NIR PBM LEDs 12 can be 30 Hz, 60 Hz, or 90 Hz, as well as any frequency at or exceeding approximately 100 Hz. If multiple imaging equipment frames rates are of concern, e.g. 24, 30) and 60 fps which may all be used in commonly available imaging equipment such as smartphones and digital cameras, then a suitable pulse frequency for pulsing the PBM LEDs 12 would be 120 Hz, although 100 Hz or above may also be used.

For the present invention, irradiation intensity refers to the delivered density of optical radiation at a target surface, such as the skin of a human subject. The irradiation intensity is described in units of optical power per square area, such as milliwatts per square centimeter (mW/cm²). The delivered dose is the cumulative product of the irradiated intensity in the target area times the duration(s) of the delivered irradiation. The delivered dose is measured in units of energy per square area, such as joules per square centimeter (J/cm²). For a PBM effect, the range of R and/or NIR irradiated intensity levels perpendicular at a target surface is preferably between 0.4 to 50 mW/cm², and more preferably between 1 to 15 mW/cm². The range of dosing per day is preferably between 0.01 to 50 J/cm², and more preferably between 0.1 to 10 J/cm², which can be spread over an eight hour period.

Systems for delivering free space radiation for PBM can take a wide variety of forms. In one form the PBM LEDs 12 are incorporated within a luminaire, that might otherwise be used for illuminating an area for general lighting purposes. For example, the luminaire may be a lighting fixture installed in a ceiling. Alternatively, the luminaire may be a lamp fixture which is located on the floor or on the surface of furniture within a built environment. In some cases the PBM LEDs 12 are integrated with illumination LEDs 11 (or other light sources) so that they are mounted in proximity to one another. In other cases, the PBM LEDs 12 and the illumination sources 11 may be in separate areas, and may be arranged to deliver different radiation patterns according to the target application. This is important because the radiation patterns typical for general illumination, and the radiation patterns useful for PBM, might be quite different.

For example, consider the case of a troffer luminaire within a hospital post-operative room or intensive care unit, and mounted above a hospital bed. An example of such a troffer luminaire is shown in FIG. 4. The troffer luminaire 40 may be mounted at the ceiling, and uses white LEDs 41 for general illumination purposes. The illumination LEDs 41 (not visible in the drawing) are positioned behind a first reflector 42 that reflects light from the LEDs up towards an additional reflective surface 43 that reflects the illumination light back downwards to light the room. The realized radiation pattern of the white illumination light from the luminaire is more or less a Lambertian, or so called “cosine” distribution 47. This broad distribution of illumination light helps illuminate large areas within a built environment, which is useful for performing general tasks in the lighted environment.

Also included in this troffer luminaire 40 are PBM LEDs 45 emitting in the R and/or NIR spectrum. The PBM LEDs 45 are not designed to throw their radiation in all directions as for general illumination from white LEDS 41, but rather to direct their radiation towards a target area, such as the hospital bed directly beneath the luminaire. The targeted, narrower radiation pattern 48 allows the PBM LEDs 45 to achieve a higher irradiation intensity for a subject on the hospital bed, compared to the irradiation intensity achieved if the PBM LEDs 45 employed the same broad distribution as the illumination LEDs 41.

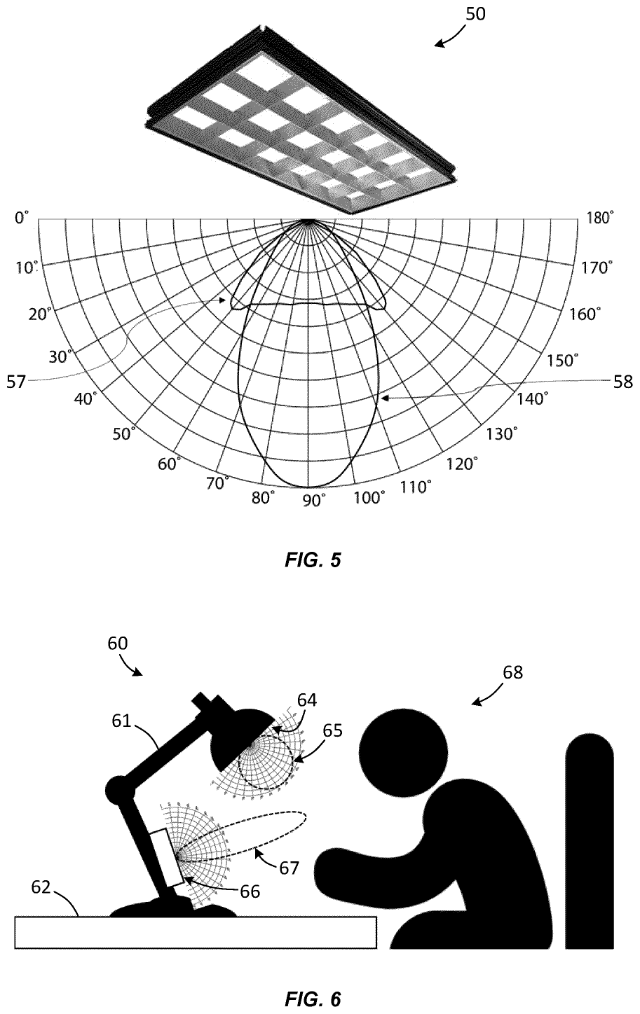

FIG. 5 illustrates another example of a troffer luminaire including white LEDs for general illumination and PBM LEDs, but in the case of a parabolic troffer luminaire 50, which can have an even wider radiation pattern, a so-called “batwing” distribution 57, for the illumination LEDs (not shown in the drawing). The PBM LEDs (not shown in the drawing) can have a narrow, more targeted radiation pattern 58 to efficiently deliver a meaningful PBM dose to a target subject at a specified location.

FIG. 6 illustrates a PBM application 60 employing a desktop lamp 61. In this particular example, the illumination LEDs 64 are incorporated into the head of the lamp, and provide a broad radiation pattern 65 useful for an illuminating a large fraction of a work surface 62 (e.g. the desktop surface) as well as a portion of the area beyond the work surface. The PBM LEDs 66, on the other hand, are included in a separate module at the shaft of the lamp, and are arranged with optics so that their delivered radiation pattern 67 is much narrower than that of the illumination LEDs 64. The radiation pattern 67 of the PBM LEDs 66 is designed to deliver a meaningful dose of PBM radiation to a target subject 68 sitting at the desk. That is, the PPM LEDs 66 will be targeting exposed skin of the human subject 68, such as the hands, face, eyes and neck, and possibly arms, forearms, and shoulders. The narrow radiation pattern 67 for the PBM LEDs 66 helps efficiently deliver radiation to the target areas without wasting it in areas where it would not be useful.

As an example, a PBM desk lamp 61 may be designed to deliver NIR radiation to a target subject 68 sitting at the desk. For example, the desk is 100 cm away from the subject 68 and targets a 100 cm diameter wide irradiation area with a symmetric radiation pattern, giving a target FWHP of 2×26.5 degrees. To achieve greater than 1 mW/cm² at the half-power angle, the delivered instantaneous radiation should be greater than 7.8 W. The cumulative dose can be delivered in a distributed fashion, as described below; to achieve a positive PBM effect without over-dosing the target subject, and without inducing other negative effects such as image flicker.

The following embodiments can include all the applications previously described, as well as many others wherein it is desirable to incorporate one or more light sources for general illumination effect as well as a PBM effect in a lighting fixture. In such applications, it is preferable to reduce or eliminate image flicker, as described above.

The term “general illumination” as used herein refers to lighting for the purpose of raising the illumination level of a space where people live or work or are active, such as in residences, offices, commercial and industrial buildings, and also outdoor locations where people are active. It means that when a space is too dark for performing desired activities, that general illumination may be used to raise the illumination level of the space to enable such activities, providing a sufficient amount of light to achieve the desired increase in the illumination level of that space. Typical illuminance levels for general lighting are at a level of 500 lm/m², or 500 lux, which corresponds to 50 mlm/cm². This means typical irradiance levels for general illumination are about about 0.2 mW/cm², using a typical lumen equivalent of radiation of 300 lm/Wₒₚₜ for white light. The choice of light sources and lighting design for general illumination should achieve illuminance levels of this order, as specified by regional codes and standards. For example, a single lighting fixture for general lighting will typically emit white light of at least 250 lumens, for example for task lighting such as a desk lamp, or at least 500 lumens or at least 2000 lumens for lighting larger spaces. Very large spaces (e.g., warehouses or stadiums) may required lighting fixtures that emit more than 10,000 lm each.

In a first embodiment of a system 10, the pulse rate for a drive current for NIR PBM LEDs 12 is increased while the pulse width is decreased to reduce image flicker while maintaining the same, continuous PBM dose. The NIR PBM irradiation is monitored using a smartphone camera with a frame rate of 30 fps. At a high enough pulse rate, 100 Hz or higher, image flicker is no longer observed. Alternatively, the pulse rate may be chosen to be a multiple of a frame rate of an imaging device, in which case image flicker is also not observed, as listed in Table 2 below.

| | Pulse | Image |
|---|---|---|
| | Rate | Flicker |
| Pulse Width (ms) | (Hz) | Perceived |
| 8.000 (comp. ex.) | 10 | Yes |
| 4.000 (comp. ex.) | 20 | Yes |
| 3.200 (comp. ex.) | 25 | Yes |
| 2.667 (Emb. 1) | 30 | No |
| 1.600 (comp. ex.) | 50 | Yes |
| 1.000 (comp. ex.) | 80 | Yes |
| 0.800 (Emb. 1) | 100 | No |
| 0.250 (Emb. 1) | 320 | No |
| 0.125 (Emb. 1) | 640 | No |

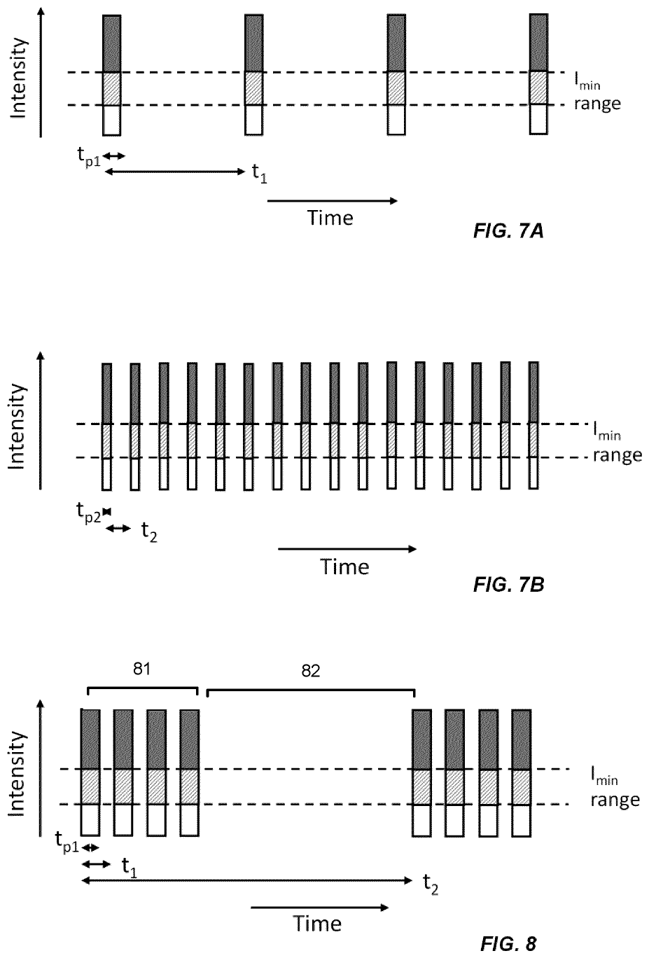

Pulse trains associated with the instantaneous intensity of PBM LEDS according to this embodiment are shown in FIG. 7. FIG. 7A shows a pulse train associated with a comparably lower frequency pulse-rate (with pulse width tₚ₁ and pulse period t₁), and unacceptable image flicker, while FIG. 7B corresponds to a higher pulse rate (with smaller pulse width tₚ₂ and smaller pulse period t₂), which can be selected to avoid image flicker as described above. Because the cumulative intensity above the threshold range for the PBM effect (indicated by the minimum irradiated intensity threshold Iₘᵢₙ in the figures) is constant, the dose delivered by each pulse train is the same. This is true as long as the duty factor, tp/t (where tp is the individual pulse width, and t is the pulse period), is kept constant.

In a second embodiment of a system 10, the choice of pulse rate for the R/NIR PBM LEDs is chosen to be a multiple of the frame rate of the imaging equipment used in the vicinity of the PBM luminaires. For example, for imaging equipment utilizing a frame rate of 30 frame per second (fps), the PBM LED pulse frequency may be chosen to be a multiple of 30 fps, such as 30 Hz, 60 Hz, or 90 Hz. Alternatively, for imaging equipment utilizing a frame rate of 25 frame per second (fps), the PBM LED pulse frequency may be chosen to be a multiple of 25 fps, such as 25 Hz, 50 Hz, 75 Hz or 100 Hz.

It is possible that, if the PBM pulse-width is too short, the desired PBM effect may not be properly triggered. In particular this is a concern at pulse widths below 1 ms. This creates a challenge for continuous-dosing solutions such as embodiments 1 and 2, since maintaining a fixed pulse width and increasing frequency increases dose proportionally, running the risk of potentially over-dosing target subjects.

In such a situation, lowering the irradiated intensity could be considered an option to reduce the total dose to a target level. However, if the PBM irradiated intensity falls below a certain threshold (as indicated by the minimum intensity range in FIG. 7 and other figures), the efficacy of the treatment may be at risk. Preferably, the irradiation intensity at a target subject's skin surface is 1 mW/cm² or greater. Simply reducing PBM LED intensity may not be an effective way to compensate for over-dosing in the case of high-frequency pulse trains with pulse widths long enough to ensure a PBM effect.

In a third embodiment of a system 10, as an alternative to continuous dosing, distributed PBM dosing may be applied. In this approach, the pulse rate of the driving current of the PBM LEDS 12 is increased enough to eliminate concern over image flicker, but the total dose for the duration-of-interest (e.g., eight hour work-shift) is managed by providing the dose in bursts, rather than continuously. This is illustrated in FIG. 8. The length of the pulse bursts 81 and the period between bursts 82 can be chosen appropriately for the application. In the lighting system 10 according to the third embodiment, the current generated by the driving circuit 17 for driving the PBM LEDs 12 may include multiple pulses during a first time period 81 (having multiple pulses with pulse width tₚ₁ and pulse period t₁) and no pulses of current during a second time period 82. The first period 81 with pulses and the second period 82 with no pulses preferably alternate with each other in a continuing fashion, over a burst cycle period t₂, during the period of PBM dosing. The pulsed current may have a predetermined pulse frequency and duty cycle during the first period 81. The first pulse frequency may be set to 100 Hz or higher, and the first duty cycle may be set to 0.5 percent or above.

For example, if the total (continuous pulse-train) PBM dose needs to be reduced by ten times, then the driving current of the PBM LEDs 12 may be pulsed so that the pulses occur in bursts, e.g. a burst of pulses may be provided lasting for 1 second of every 10 seconds, or 10 seconds every 100 seconds, or 1 minute every 10 minutes, etc. In this approach, the pulse train is simply turned-off at appropriate intervals, in order to maintain a desired dose over the total duration-of-interest. This is illustrated below in Table 3, showing examples related to embodiment 3, with pulse-width of 8 ms and pulse-rate of 100 Hz. PBM irradiation intensity is fixed and chosen to provide an optimal dose over 8 hours in continuous mode at 8 ms per 100 ms.

| | Pulse | On | Off | | | |
|---|---|---|---|---|---|---|
| | Rate | Period | Period | On | Relative | |
| | (Hz) | (s) | (s) | Fraction | Dose | Comment |
| Comp. ex. | 10 | cont. | 0 | 100% | 1x | Image flicker |
| Comp. ex. | 100 | cont. | 0 | 100% | 10x | Dose too high |
| Emb. 3A | 100 | 1 | 9 | 10% | 1x | |
| Emb. 3B | 100 | 10 | 90 | 10% | 1x | |
| Emb. 3C | 100 | 100 | 900 | 10% | 1x | |

The choice of on/off periods should be reasonable for the application. For example, considering an eight-hour shift, it may be preferred to distribute the pulse bursts relatively uniformly over the eight-hour period. For example, for a 10%“on-fraction”, having pulse bursts every 6 seconds out of every 1 minute, 1 minute out of 10 minutes, or 6 minutes every hour, has the desirable outcome that most target subjects interacting within the irradiated area are likely to experience a meaningful PBM dose near the optimum level. This is in contrast to the case wherein the pulse bursts are applied in a singular 48 minute period within the eight-hour duration. If a target subject should have left the irradiated area, for whatever reason, during this time, they would experience a sub-optimal PBM dose, or potentially no dose at all.

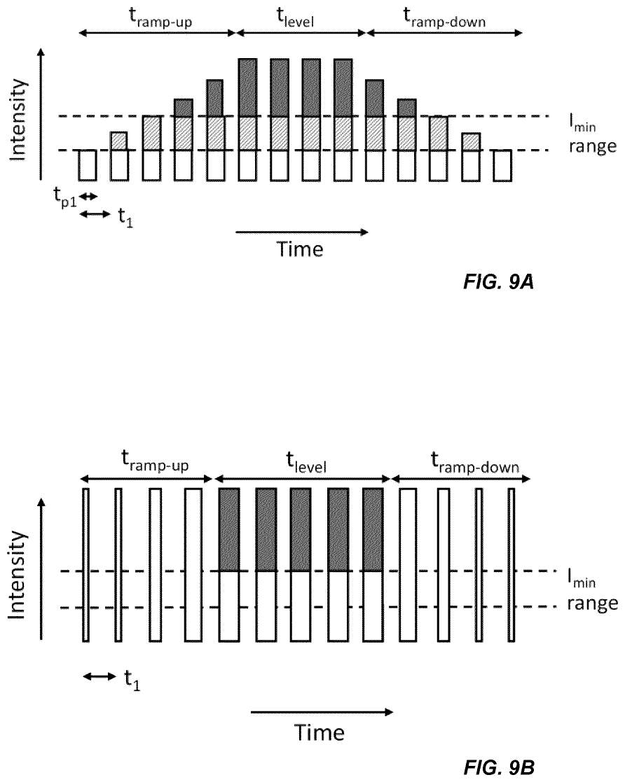

A potential problem for the previous embodiment may be that the sudden turn-on, or turn-off, of the pulse burst may be noticeable to bystanders or those operating imaging equipment. To address this issue, a fourth embodiment of a system 10 provides for a “phase in” of the burst of pulses, so that the gradual increase in irradiation level during each pulse burst is not perceived by target subjects or others in the area, and is not objectionable to operators of digital imaging equipment whose auto-leveling features can deal with such changes, provided they happen over a slow enough time scale. This embodiment is illustrated in FIG. 9A, showing one pulse burst, which will be repeated following a period of no pulses to form alternating first periods with pulse bursts and second periods without pulses, over a burst cycle period, similarly to the third embodiment.

The ramp-up time period tᵣₐₘₚ₋ᵤₚ and ramp-down time period t_{ramp-down} may be slow enough to avoid notice by end-users. The level time period tₗₑᵥₑₗ when the pulses are at their maximum value and total on-period are chosen to generate the targeted PBM dose during pulse bursts, such that the sum total of the bursts over the PBM application duration reach the total target PBM dose level.

In the lighting system 10 according to the fourth embodiment, the driver circuit 17 is adapted to provide a pulsed current to the PBM LEDs 12 having an amplitude of the pulses which increases for successive ones of the pulses during a first portion of the first period (tᵣₐₘₚ₋ᵤₚ), and decreases for successive ones of the pulses during a last portion of the first period (t_{ramp-down}). The driver circuit may be adapted to generate the first pulsed current having an amplitude of the pulses which is substantially constant during a second portion of the first period (tₗₑᵥₑₗ).

The choices of durations for ramp-up, level, and ramp-down time periods are preferably selected so that the existence of PBM irradiation is not detected by end-users or by imaging equipment. Specific choices will be determined by detailed product requirements and overall target dose, but generally it is desired that a ramp-up and ramp-down time periods be at least 10 seconds, preferably 1 minute or more, and more preferably 5 minutes or more.

A fifth embodiment of a system 10 utilizes pulse-width modulation (PWM) to slowly ramp-up to a PBM dose level. In this embodiment, once a turn-on trigger condition is met, a “phase in” of pulse widths is provided, starting from a very low duty factor, so that a gradual increase in dose level is achieved during each pulse as pulse duration is systematically increased. When the target dose velocity is achieved, the pulse width is kept constant. Once the cumulative target dose is achieved, the pulse width is systematically reduced and eventually the pulse train is turned off, until the next cycle is ready to begin. When the pulse frequency is high enough (preferably above 100 Hz), the human eye will average out the effects and simply perceive (if at all) a slow, gradual increase in deep red irradiation (similar to the case for FIG. 9A), which is not perceived as objectionable by target subjects or others in the area, and is not objectionable to operators of digital imaging equipment whose auto-leveling features can deal with such changes, provided they happen over a slow enough time scale. This embodiment is illustrated in FIG. 9B, showing one pulse burst, which will be repeated following a period of no pulses to form alternating first periods with pulse bursts and second periods without pulses, over a burst cycle period, similarly to the third embodiment.

In the lighting system 10 according to the fifth embodiment, the driver circuit 17 is adapted to provide a pulsed current to the PBM LEDs 12 where the pulse width tₚ of the pulses increases for successive ones of the pulses during a first portion of the first period (tᵣₐₘₚ₋ᵤₚ), and decreases for successive ones of the pulses during a last portion of the first period (t_{ramp-down}). The driver circuit may be adapted to generate the first pulsed current having a pulse width of the pulses which is substantially constant during a second portion of the first period (tₗₑᵥₑₗ).

The choices of durations for ramp-up, level, and ramp-down time periods are preferably selected so that the existence of PBM irradiation is not detected by end-users or by imaging equipment. Specific choices will be determined by detailed product requirements and overall target dose, but generally it is desired that a ramp-up and ramp-down time periods be at least 10 seconds, preferably 1 minute or more, and more preferably 5 minutes or more.

In the limit case of embodiments 4 and 5, wherein the duty factor for the pulses reaches 100%, the PBM radiation sources are no longer high-frequency pulsed, but simply running in a continuous-wave (cw) mode during the ramp-up, level, and ramp-down time periods. In a fifth embodiment of a system 10, this option is implemented and provides the advantage of more simplified signal generator and driver circuits 15, 17 compared to the high-frequency pulses embodiments. However, as in all embodiments, care must be taken to properly heatsink and thus manage the thermal environment of the radiation sources, especially in the case of R and NIR LEDs, which are notoriously temperature sensitive.

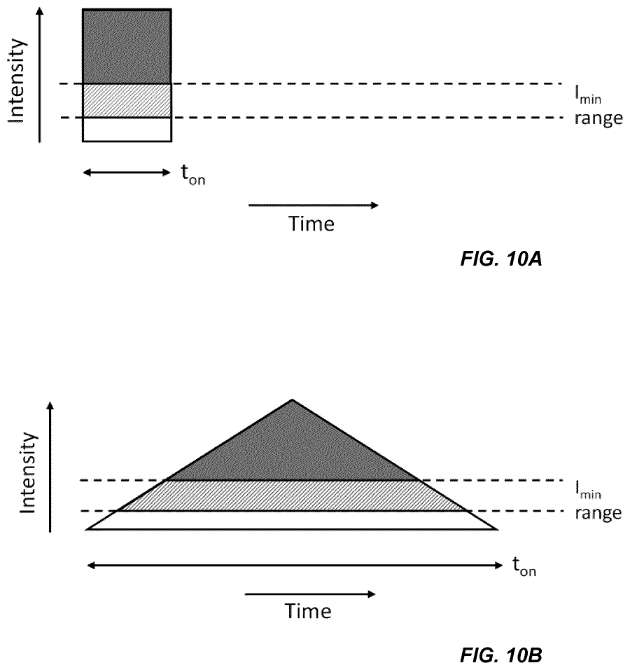

FIG. 10 shows irradiation intensity profiles for examples utilizing continuous wave (cw) operation of the PBM radiation sources. The top profile in FIG. 10A shows a simple on/off burst-mode using cw operation. The bottom profile in FIG. 10B shows a ramp-up/ramp-down burst using cw operation. As for the other embodiments, the intensity profiles are chosen such that the total doses (above the minimum irradiated intensity threshold, Iₘᵢₙ) are the same.

In the lighting system 10 according to the sixth embodiment, the driver circuit 17 is adapted to provide a current to the PBM LEDs 12 during a first period and not during a second period, the first period and the second period alternating with each other, and the driver circuit is configured to gradually increase the amplitude of the first current during a first portion of each first period, and gradually decrease the amplitude of the first current during a last portion of each first period. The driver circuit 17 may be configured to maintain the first current at a substantially constant amplitude during a second portion of each first period, the second portion occurring between the first portion and the last portion of each first period.

### Examples

The data shown below illustrates an example of design parameters and calculations that can be applied for any of the disclosed embodiments, using techniques familiar to the person of ordinary skill in the art. This example is based on the design distance from the PBM light source to the target surface, desired radiation intensity at the target surface to generate the PBM effect, and target diameter of the irradiated area on the target surface. The half-power angle and peak and average power required for the PBM LEDs, and number of PBM LEDs and crest factor may be determined.

| | Target Diameter (m) | | | | | | | |
|---|---|---|---|---|---|---|---|---|
| | 0.2 | 0.5 | 1 | 2 | | | | |
| Distance (m) | Half-Power Angle (deg) | | | | | | | |
| 0.2 | 27 | 51 | 68 | 79 | | | | |
| 0.5 | 11 | 27 | 45 | 63 | | | | |
| 1 | 5.7 | 14 | 27 | 45 | | | | |
| 2 | 2.9 | 7.1 | 14 | 27 | | | | |
| Irradiance | | | | | | | | |
| (mW/cm2) | PBM Peak Power Required (W) | | | | PBM Avg Power Required (W) | | | |
| 1 | 0.6 | 3.9 | 15.7 | 62.8 | 0.13 | 0.79 | 3.14 | 12.6 |
| 3 | 1.9 | 11.8 | 47.1 | 188.5 | 0.38 | 2.36 | 9.4 | 37.7 |
| 10 | 6.3 | 39.3 | 157.1 | 628.3 | 1.26 | 7.9 | 31.4 | 125.7 |
| 30 | 18.8 | 117.8 | 471.2 | 1885 | 3.77 | 23.6 | 94.2 | 377 |
| Pulse Width (ms) | | | | | No. of LEDs Required | | | |
| 2 | | | | | 1 | 1 | 3 | 10 |
| Frequency (Hz) | | | | | 1 | 2 | 7 | 28 |
| 100 | | | | | 1 | 6 | 24 | 94 |
| Duty Factor | | | | | 3 | 18 | 70 | 280 |
| 20% | | | | | Crest Factor Required | | | |
| Power per LED (W) | | | | | 0.5 | 2.9 | 3.9 | 4.7 |
| 1.35 | | | | | 1.4 | 4.4 | 5.0 | 5 |
| | | | | | 4.7 | 4.8 | 4.8 | 5.0 |
| | | | | | 4.7 | 4.8 | 5 | 5 |

For these design parameters, the dose at the subject target for providing PBM effect may be calculated for various exposure durations, and the “on-time” fraction calculated, i.e. the percentage of time when the PBM LEDs are energized.

| | Duration (h) | | | | | | Target Dose | |
|---|---|---|---|---|---|---|---|---|
| | 1 | 2 | 4 | 8 | | | 1 | |
| Irradiance | | | | | | | | |
| (mW/cm2) | Dose, continuous PBM (J/cm2) | | | | Distributed Dosing “on-time” fraction | | | |
| 1 | 0.7 | 1.4 | 2.9 | 5.8 | 100% | 69% | 35% | 17% |
| 3 | 2.2 | 4.3 | 8.6 | 17 | 46% | 23% | 12% | 5.8% |
| 10 | 7.2 | 14 | 29 | 58 | 14% | 6.9% | 3.5% | 1.7% |
| 30 | 22 | 43 | 86 | 173 | 5% | 2.3% | 1.2% | 0.6% |

As an example, a PBM desk lamp may be designed to deliver NIR radiation to a target subject sitting at the desk. The desk lamp is approximately 100 cm away from the subject and targets a 100 cm diameter wide irradiation area with a symmetric radiation pattern, giving a target FWHP of 2×26.5 deg. To achieve greater than 1 mW/cm² at the half-power angle, the delivered instantaneous (or, peak) irradiation should be greater than 7.8 W, such that the total emitted power from the PBM LEDs will be twice that, or 15.7 W. The PBM pulse frequency may be chosen to be 100 Hz, to avoid image flicker problems. The PBM pulse width may be 2 ms, which gives a duty factor of 20%. Thus, the average PBM power delivery will be 3.14 Watts. This is possible using three NIR LEDs, such as the commercially available LUXEON IR Domed for Automotive Line L1I0-A850050000000 product from Lumileds. These devices are capable of 1.35 W for 1 A per emitter in continuous, dc, mode. Three of them can deliver ˜4 W, requiring a “crest factor” (i.e., ratio of achievable peak output power to continuous dc power) of about 4, which is well achievable given the datasheet suggestion of maximum pulsed current of 5 A (i.e., crest factor of 5 A/1 A=5).

Under these conditions the desk lamp would deliver a dose of 5.8 J/cm² to the target area over an eight hour period. It may be desirable to target a lower dose. For example, a overall dose of 1 J/cm² might be targeted for an eight-hour period. In this case, distributed dosing, as described in more detail below, might be employed. For example, the PBM desk lamp could target on overall “on-time” fraction of 17%, or about 10 minutes each hour, for example, to deliver the target cumulative dose. The PBM lamp turn-on might be in a simple on/off fashion, or phased in slowly over time to avoid conspicuous detection by end-users or by imaging equipment in use in proximity to the target area. For example, the PBM lamp radiation might be linearly ramped from 0 W to the peak 15.7 W over a 5 minute time period, then ramped back down later over a similar time period, such that the total cumulative dose target is achieved. Obviously, many other combinations and specifications are possible within a detailed product development framework, but all of which fall within the scope of the teachings of the present invention.

It is evident that the various embodiments described and explained above are mutual compatible with each other, unless explicitly stated. As such, the combination of any number of the features from the above embodiments is still within the present disclosure. For example, different combinations of exemplary predetermined spectrums, exemplary (peak) emission power levels of the radiation source and exemplary brightness of the light source are clearly within the scope of the present disclosure. Additionally, the features in the above embodiments may be disclaimed or otherwise left out.

1. A lighting system comprising:
a first light source adapted to emit light substantially only in a first predetermined spectrum in a range from 600 nm to 1400 nm; and
a driver circuit arranged to provide a first pulsed current to the first light source for producing the light in the first predetermined spectrum;
wherein the driver circuit is adapted to generate multiple pulses of the first pulsed current during a first period and no pulses of current during a second period, the first period and the second period alternating with each other; and
wherein the first pulsed current has a first pulse frequency and a first duty cycle during the first period, the first pulse frequency being 100 Hz or higher, and the first duty cycle being 0.5 percent or above.

2. The lighting system of claim 1, wherein the driver circuit is adapted to generate the first pulsed current having an amplitude of the pulses which increases for successive ones of the pulses during a first portion of the first period, and decreases for successive ones of the pulses during a last portion of the first period.

3. The lighting system of claim 2, wherein the driver circuit is adapted to generate the first pulsed current having an amplitude of the pulses which is substantially constant during a second portion of the first period.

4. The lighting system of claim 1, wherein the driver circuit is adapted to generate the first pulsed current having a pulse-width of the pulses which increases for successive ones of the pulses during a first portion of the first period, and decreases for successive ones of the pulses during a subsequent portion of the first period.

5. The lighting system of any one of the preceding claims, wherein the driver circuit is adapted to generate the first pulsed current having a pulse frequency which is a multiple of 24 pulses per second and/or 30 pulses per second.

6. The lighting system of any one of the preceding claims, wherein the driver circuit is adapted to generate the first pulsed current having a pulse frequency which is a multiple of a mains power supply frequency.

7. The lighting system of any one of the preceding claims, wherein the driver circuit is adapted to generate the first pulsed current having a pulse frequency being a multiple of a frame rate of an imaging device capable of recording images and/or video.

8. The lighting system of any one of the preceding claims, wherein the driver circuit is adapted to generate the first pulsed current having a width of the pulses of 0.05 ms or more.

9. The lighting system of any one of the preceding claims, wherein the driver circuit is adapted to generate the first pulsed current having a period between pulses of 0.05 ms or more.

10. A lighting system comprising:
a first light source adapted to emit light substantially only in a first predetermined spectrum in a range from 600 nm to 1400 nm;
a driver circuit adapted to provide a first current to the first light source for producing the light in the first predetermined spectrum;
wherein the driver circuit is configured to provide the first current during a first period and not during a second period, the first period and the second period alternating with each other;
wherein the driver circuit is configured to gradually increase the amplitude of the first current during a first portion of each first period, and gradually decrease the amplitude of the first current during a last portion of each first period.

11. The lighting system of claim 10, wherein the driver circuit is configured to maintain the first current at a substantially constant amplitude during a second portion of each first period, the second portion occurring between the first portion and the last portion of each first period.

12. The lighting system of any one of the preceding claims, wherein ratio between the first period to the second period is 1:10 or less.

13. The lighting system of any one of the preceding claims, wherein the irradiation intensity at an average distance of between 0.2 and 5 m from the first light source is 1 mW/cm² or more, preferably between 0.4 and 50 mW/cm², and more preferably between 1 and 15 mW/cm².

14. The lighting system of any one of the preceding claims, wherein the irradiation intensity at an average distance of between 0.2 and 5 m from the first light source is sufficient to induce a photobiomodulation effect in a human.

15. The lighting system of any one of the preceding claims, wherein the delivered dose over 8 hours at an average distance of between 0.2 and 5 m from the first light source is between 0.01 and 50 J/cm², and preferably between 0.1 and 10 J/cm².

16. The lighting system of any one of the preceding claims, further comprising a second light source adapted to emit white light suitable for general illumination, wherein the second light source is adapted to emit at least 250 lumens, preferably at least 1000 lumens, more preferably at least 2000 lumens when operating.

17. The lighting system of claim 15, wherein the white light emitted by the second light source is directed onto one or more reflectors so that the white light is emitted from the lighting system having a radiation pattern with a full-width-at-half-power angle of 2×23 degrees or more.

18. The lighting system of any one of the preceding claims, wherein the light emitted by the first light source is emitted from the lighting system having a radiation pattern with a full-width-at-half-power angle of 2×45 degrees or less.

19. The lighting system of any one of claims 16-18, comprising a luminaire, wherein the first and second light sources and the one or more reflectors are installed in the luminaire.

20. The lighting system of any one of claims 16-19, comprising a lamp for lighting a work space, wherein the first and second light sources and the one or more reflectors are mounted in the lamp, the lamp being adapted to direct the white light from the second light source onto a work space and to direct the light from the first light source onto a user.
